(19) United States
(12) Patent Application Publication
(10) Pub. No.: US 2014/0031564 A1
(43) Pub. Date: Jan. 30, 2014

(54) PROCESS FOR PREPARING (S)-3-N-METHYLAMINO-1-(2-THIENYL)-1-PROPANOL

(71) Applicants: Michael Kreis, Leverkusen (DE); Antje Steinhaus, Koeln (DE); Guido Giffels, St. Augustin (DE); Oliver Krebs, Langenfeld (DE)

(72) Inventors: Michael Kreis, Leverkusen (DE); Antje Steinhaus, Koeln (DE); Guido Giffels, St. Augustin (DE); Oliver Krebs, Langenfeld (DE)

(73) Assignee: SALTIGO GmbH, Langenfeld (DE)

(21) Appl. No.: 13/819,447

(22) PCT Filed: Aug. 26, 2011
(86) PCT No.: PCT/EP2011/064700

§ 371 Date: Oct. 11, 2013

(30) Foreign Application Priority Data
Aug. 30, 2010 (EP) 10174550.3

(51) Int. Cl. C07D 333/20

(52) U.S. Cl. CPC C07D 333/20
USPC 549/75

(57) ABSTRACT

The present invention relates to a method for preparing (S)-3-N-methylamino-1-(2-thienyl)-1-propanol and salts thereof.

The present invention relates to a method for preparing (S)-3-N-methylamino-1-(2-thienyl)-1-propanol and salts thereof.

(S)-3-N-methylamino-1-(2-thienyl)-1-propanol is an important intermediate in the synthesis of the bioactive substance (S)-N-methyl-3-(1-naphthyloxy)-3-(2-thienyl)propanamine hydrochloride (Duloxetine). (S)-N-methyl-3-(1-naphthyloxy)-3-(2-thienyl)propanamine hydrochloride acts as a serotonin reuptake inhibitor and is used as an anti-depressant and also for the treatment of incontinence (Hulling et al. Chirality, 2000, 12, 26-29 and Sorbera, Drugs of the Future, 2000, 25, 907-916).

WO 2004031168 describes the preparation of (S)-3-N-methylamino-1-(2-thienyl)-1-propanol by asymmetric transfer hydrogenation of (S)-3-N-methylamino-1-(2-thienyl)-1-propanone using (1S,2R)-(-)-cis-1-amino-2-indanol as chiral ligand and (p-cymene)ruthenium(II) dimer as catalyst precursor. This method is disadvantageous since only small enantiomeric excesses are achieved and a diastereoselective crystallisation using an expensive chiral acid as an additional reaction step is therefore required.

It is known from EP 1510517 A, Angew. Chem. Int. Ed., 2005, 44, 1687 and WO 2004020389 that (S)-3-N-methylamino-1-(2-thienyl)-1-propanol can be prepared by assymetric hydrogenation of 3-N-methylamino-1-(2-thienyl)-1-proparione using chiral bidentate phosphine ligands. WO 2006087166 describes a method for preparing (S)-3-N-methylamino-1-(2-thienyl)-1-propanolsulphonic acid salts by asymmetric hydrogenation of 3-N-methylamino-1-(2-thienyl)-1-propanonesulphonic acid salts also using chiral bidentate phosphine ligands. Common to the methods is that they are inefficient industrial processes, since expensive phosphine ligands are used and complex equipment is required due to the hydrogenation with hydrogen under high pressure.

A further method for preparing (S)-3-N-methylamino-1-(2-thienyl)-1-propanol by enzymatically catalysed reduction of 3-N-methylarnino-1-(2-thienyl)-1-propanone is disclosed in WO 2004065376. The microorganisms employed are difficult to cultivate such that the method cannot be efficiently carried out on an industrial scale.

WO 2004011452 describes the asymmetric reduction of 3-N-methylarnino-1-(2-thienyl)-1-propanone to (S)-3-N-methylamino-1-(2-thienyl)-1-propanol using ruthenium catalysts and chiral, bidentate phosphine ligands and diamine ligands which can be carried out similarly by transfer hydrogenation. Disadvantages of this method are that the enantiomeric purities and yields achieved are insufficient.

A method for preparing 3-N-methylamino-1-(2-thienyl)-1-propanone by asymmetric transfer hydrogenation of 0-ketoacid esters to f3-hydroxyacid esters, particularly to give (S)-methyl 3-hydroxy-3-(2-thienyl)propanoate, and further reduction to 3-N-methylarnino-1-(2-thienyl)-1-propanone, is known from EP 1340746. Since expensive reducing agents, having safety-related concerns, must be used, such as lithium aluminum hydride, this method cannot be efficiently carried out on an industrial scale.

Therefore, there still exists a need for a method for preparing (S)-3-N-methylamino-1-(2-thienyl)-1-propanol which can overcome the disadvantages of the prior art, and where (S)-3-N-methylamino-1-(2-thienyl)-1-propanol can be prepared with high enantiomeric purities and in good yields.

Surprisingly a method has been found, based on an asymmetric transfer hydrogenation, with which the disadvantages of the prior art could be overcome, and (S)-3-N-methylamino-1-(2-thienyl)-1-propanol can be prepared in an economic and efficient manner, in high enantiomeric purities and good yields on an industrial scale.

The present invention therefore provides a method for preparing (S)-3-N-methylamino-1-(2-thienyl)-1-propanol and salts thereof, by reacting 3-N-methylamino-1-(2-thienyl)-1-propanone or salts thereof in the presence of at least one compound of formula (I)

[RuX₂(arene)]₂ (I)

where arene is an aromatic compound, bound to ruthenium in a co-ordinated manner, having 6 to 12 ring carbon atoms, which may be further substituted with up to 6 radicals, which independently of one another are selected from the group consisting of C₁-C₈-alkyl, benzyl and phenyl, and X is chlorine, bromine or iodine,

in the presence of at least one compound of formula (II)

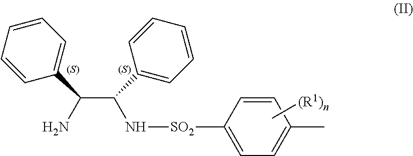

where R¹ can be the same or different and each is a radical from the series C₁-C₂₀-alkyl, C₁-C₂₀-alkoxy, C₁-C₂₀-fluoroalkyl, halogen, or C₆-C₁₅-aryl and n is 0, 1, 2, 3 or 4,

in the presence of at least one amine and

in the presence of formic acid or/and formates or mixtures thereof.

Arene is preferably benzene or naphthalene, which may be substituted with up to 6 radicals, which independently of one another are selected from the group consisting of methyl, ethyl, n-propyl, isopropyl and tert-butyl.

Arene is particularly preferably mesitylene, cumene, p-cymene, o-cymene, m-cymene or benzene. Arene is especially preferably p-cymene.

X is preferably chlorine.

Compounds of formula (I) are particularly preferably benzenedichlororuthenium dimer, mesitylenedichlororuthenium dimer and cumenedichlororuthenium dimer, o-cymene-dichlororuthenium dimer, m-cymenedichlororuthenium dimer or p-cymenedichlororuthenium dimer where p-cymenedichlororuthenium dimer, also referred to as di-n-chlorobis[chloro(p-cymene)ruthenium(II)] is more particularly preferred.

n is preferably 0, 1, 2 or 3. With particular preference, n is 0 or 1. Particular preference is given to n=0.

R¹ is preferably C₁-C₄-alkyl, C₁-C₄-fluoroalkyl, C₁-C₄-alkoxy, phenyl or fluorine and chlorine.

R¹ is with particular preference methyl, ethyl, n-, isopropyl, n-, sec-, tert-butyl or n-, sec-pentyl or fluorine or chlorine.

R¹ is even more preferably methyl.

The compound of formula (11) is particularly preferably S,S-N-p-toluenesulphonyl-1,2-diphenylethylenediarnine and the compound of formula (1) p-cymenedichlororuthenium dimer.

In the context of the invention, amines are cyclic, branced or unbranched mono-, di-, or tertiary alkylamines or mono-, di-, or tertiary arylamines or mixtures of these amines. Examples of amines are methylamine, ethylamine, propylamine, isopropylamine, triethylamine, trimethylamine, triphenylamine, tripropylamine, tripentylamine, n-butylamine, isobutylamine, tert-butylamine, cyclohexylamine, cyclooctylamine, pentylamine or octylamine. With preference, the amine is a tertiary alkyl- or arylamine, such as triethylamine, trimethylamine, triphenylamine, tripropylamine, tripentylamine or tert-butylamine. With particular preference, the amine is a tertiary alkylamine. Still more preferred is the amine triethylamine.

The scope of the invention encompasses all the stated radical definitions, parameters and illustrations, in general or in preferred ranges, mentioned above and in the following, with respect to one another, thus also between the respective ranges and preferred ranges in any combination.

C₁-C₆-alkoxy is, for example, methoxy, ethoxy, n-propoxy, isopropoxy, n-butoxy, sec-butoxy and tert-butoxy, n-pentoxy, 1-methylbutoxy, 2-methylbutoxy, 3-methylbutoxy, neopentoxy, 1-ethylpropoxy, cyclohexoxy, cyclopentoxy and n-hexoxy.

C₁-C₄-fluoroalkyl is preferably fluoromethyl, difluoromethyl, trifluoromethyl, fluoroethyl, difluoroethyl, 2,2,2-trifluoroethyl, tetrafluoroethyl, pentafluoroethyl, fluoropropyl, heptafluoropropyl, heptafluoroisopropyl, hexafluoroisopropyl and nonafluorobutyl.

Halogen is preferably chlorine or fluorine. Fluorine is particularly preferred.

The method according to the invention may be carried out in the presence or absence of solvents. The method is preferably carried out in the presence of organic solvent.

Particularly suitable solvents for carrying out the method according to the invention are amides, such as dimethylformamide, N-methylpyrrolidinone, aliphatic or aromatic, optionally halogenated solvents having up to 16 carbon atoms such as toluene, o-, p-xylene, chloroform, dichloromethane, chlorobenzene, the isomeric dichlorobenzenes, fluorobenzene, nitriles such as acetonitrile, benzonitrile, dimethyl sulphoxide or alcohols, such as methanol, ethanol, propanol or butanol, or mixtures of said solvents.

Preferred solvents are amides, nitriles or alcohols or mixtures of these solvents. As solvent, particular preference is given to the use of acetonitrile, dimethylformamide, dimethylacetamide, N-methylpyrrolidinone or methanol or mixtures of these solvents.

Mixtures of formic acid with amines are preferably used. In so doing, the corresponding ammonium formates at least partially form, which may of course be used in an analogous manner.

The molar ratio of formic acid to tertiary amine may preferably be, for example, 10:1 to 1:10, particularly preferably 3:1 to 0.8:1.

The molar ratio of formic acid based on the substrate used is preferably 10:1 to 1:1 .

The method according to the invention may be carried out, for example, at temperatures of 0 to 100° C., preferably 20° C. to 80° C., particularly preferably at temperatures of 30° C. to 60° C.

In general, the reaction times in the inventive method may be selected as desired. The reaction times are preferably between 0.1 h and 72 h, particularly preferably between 1 h and 48 h.

The molar amount of ruthenium used in the method according to the invention can be, for example, 0.05 to 10 mol %, based on the amount of 3-N-alkylamino-1-(2-thienyl)-1-propanone, or salts thereof, used. The amount of ruthenium used in the method according to the invention is preferably 0.05 to 5 mol %, particularly preferably 0.05 to 1 mol %, based on the amount of 3-N-alkylamino-1-(2-thienyl)-1-propanone, or salts thereof, used.

The molar ratio of compounds of formula (I) to compounds of formula (II) may be, for example, 4:1 to 1:4, preferably the molar ratios are 1:1 to 1:4, particularly preferably the molar ratios of compounds of formula (I) to compounds of formula (II) in the method according to the invention are 1:1 to 1:2.

(S)-3-N-methylamino-1-(2-thienyl)-1-propanol preferably has an enantiomeric purity of 90% or more, particularly preferably of 95% or more and especially preferably of 99% or more.

The compounds of formula (I) and the compounds of formula (II) react together over a catalyst. Thus, according to the invention, a method is also included in which the catalyst in the form of compounds of formula (III) is added.

In formula (III)

[RuX₂(arene){(III)}] (III)

arene and X each have the meaning and preferred ranges given for formula (I), and (II) in formula (III) represents compounds of formula (II) having the meanings and preferred ranges stated thereunder.

Examples of compounds of formula (III) include:

[N-[(1S,2S)-2-(amino-κN)-1,2-diphenylethyl]-p-tolylsulphonamidato-κN]chloro[(η⁶)-p-cymene]-ruthenium(II)

[N-[(1S,2S)-2-(amino-κN)-1,2-diphenylethyl]-o-tolylsulphonamidato-κN]chloro[(η⁶)-p-cymene]-ruthenium(II)

[N-[(1S,2S)-2-(amino-κN)-1,2-diphenylethyl]-m-tolylsulphonamidato-κN]chloro[(η⁶)-p-cymene]-ruthenium(II)

[N-[(1S,2S)-2-(amino-κN)-1,2-diphenylethyl]phenylsulphonamidato-κN]chloro[(η⁶)-p-cymene]-ruthenium(II)

[N-[(1S,2S)-2-(amino-κN)-1,2-diphenylethyl]-4-ethylphenylsulphonamidato-κN]chloro[(η⁶)-p-cymene]-ruthenium(II)

[N-[(1S,2S)-2-(amino-κN)-1,2-diphenylethyl]-3-ethylphenylsulphonamidato-κN]chloro[(η⁶)-p-cymene]ruthenium(II)

[N-[(1S,2S)-2-(amino-κN)-1,2-diphenylethyl]-2-ethylphenylsulphonamidato-κN]chloro[(η⁶)-p-cymene]ruthenium(II)

[N-[(1S,2S)-2-(amino-κN)-1,2-diphenylethyl]-2,4,6-trimethylphenylsulphonamidato-κN]chloro[(η⁶)-p-cymene]ruthenium(II)

[N-[(1S,2S)-2-(amino-κN)-1,2-diphenylethyl]-2,4,6-triisopropylphenylsulphonarnidato-κN]chloro[(η⁶)-p-cyrnene]ruthenium(II)

[N-[(1S,2S)-2-(amino-κN-1,2-diphenylethyl]-4-chlorophenylsulphonarnidato-κ]chloro[(η⁶)-p-cymene]ruthenium(II)

[N-[(1S,2S)-2-(amino-κN)-1,2-diphenylethyl]-3-chlorophenylsulphonamidato-κN]chloro[(η⁶)-p-cymene]ruthenium(II)

[N-[(1S,2S)-2-(amino-κN)-1,2-diphenylethyl]-2-chlorophenylsulphonamidato-κN]chloro[(η⁶)-p-cymene]ruthenium(II)

[N-[(1S,2S)-2-(amino-κN)-1,2-diphenylethyl]-4-fluorophenylsulphonamidato-κN]chloro[(η⁶)-p-cymene]ruthenium(II)

[N-[(1S,2S)-2-(amino-κN)-1,2-diphenylethyl]-3-fluorophenylsulphonamidato-κN]chloro[(η⁶)-p-cymene]ruthenium(II)

[N-[(1S,2S)-2-(amino-κN)-1,2-diphenylethyl]-2-fluorophenylsuiphonamidato-κN]chloro[(η⁶)-p-cymene]ruthenium(II)

[N-[(1S,2S)-2-(amino-κN)-1,2-diphenylethyl]-4-methoxyphenylsulphonamidato-κN]chloro[(η⁶)-p-cymene]ruthenium(II)

[N-[(1S,2S)-2-(amino-κN)-1,2-diphenylethyl]-3-methoxyphenylsulphonamidato-κN]chloro[(η⁶)-p-cymene]ruthenium(II)

[N-[(1S,2S)-2-(amino-κN)-1,2-diphenylethyl]-2-methoxyphenylsulphonamidato-κN]chloro[(η⁶)-p-cymene]ruthenium(II)

[N-[(1S,2S)-2-(amino-κN)-1,2-diphenylethyl]pentafluorophenylsulphonanaidato-κN]chloro[(η⁶)-p-cymene]ruthenium(II)

The compound of formula (III) used is preferably [N-[(1S,2S)-2-(amino-κN)-1,2-diphenylethyl]-p-tolylsulphonamidato-κN]chloro[(η⁶)-p-cymene]ruthenium(II).

The method according to the invention may be carried out at reduced, atmospheric or elevated pressure. Preferably, the method is carried out at atmospheric or elevated pressure, e.g. at 1 to 16 bar, particularly preferably at 1 to 10 bar. The reaction is especially preferably carried out at atmospheric pressure.

Some of the reactants and/or starting materials employed are commercially available, or may be synthesised by methods known to those skilled in the art. Thus, 3-N-methylainino-1-(2-thienyl)-1-propanone can be prepared, for example, starting from acetylthiophene, methylamine and formaldehyde by a Mannich reaction (Mannich et al. Chem. Ber. 1922, 55, 356, Blicke et al. and J. Am. Chem. Soc. 1942, 64, 451).

3-N-Methylamino-1-(2-thienyl)-1-propanone is used according to the inventive method as the compound or/and in the form of salts thereof. 3-N-Methylamino-1-(2-thienyl)-1-propanone is an amine base, which, particularly with protic acids such as hydrohalic acids, may be converted to the related acid addition salt, for example by reacting equivalent amounts of the base and the acid in an inert solvent such as ethanol and subsequent evaporation. Corresponding salts of the 3-N-methylamino-1-(2-thienyl)-1-proparione are therefore preferably, for example, sulphates, hydrogensulphates, nitrates, hydrochlorides, hydrobromides, hydrofluorides, phosphates, hydrogenphosphates, salts of organic acids such as aliphatic, alicyclic, aromatic or heterocyclic mono or polycarboxylates, e.g. formates, acetates, propionates, pivalates, diethylacetates, citrates, tartrates, gluconates or e.g. sulphonates such as methanesulphonate (mesylate), ethanesulfonate, propanesulphonate, tosylates, halosulphonates, such as triflate.

The salts used are particularly preferably aliphatic, alicyclic, aromatic or heterocyclic sulphonic acids or hydrohalic acids. The salts used are especially preferably methanesulphonate or hydrochloride.

Salts of (S)-3-N-methylarnino-1-(2-thienyl)-1-propanol are preferably, for example, sulphates, hydrogensulphates, nitrates, hydrochlorides, hydrobromides, hydrofluorides, phosphates, hydrogenphosphates, salts of organic acids such as aliphatic, alicyclic, aromatic or heterocyclic mono- or polycarboxylates, e.g. formates, acetates, propionates, pivalates, diethylacetates, citrates, tartrates, gluconates or e.g. sulphonates such as methanesulphonate (mesylate), ethanesulfonate, propanesulphonate, tosylates, halosulphonates, such as triflate.

The salts of 3-N-methylamino-1-(2-thienyl)-1-propanone employed and the salts prepared of (S)-3-N-methylamino-1-(2-thienyl)-1-propanol are preferably identical.

The invention preferably comprises a method in which 3-N-methylamino-1-(2-thienyl)-1-propanone or a salt thereof, particularly preferably 3-N-methylamino-1-(2-thienyl)-1-propanone hydrochloride or 3-N-methylamino-1-(2-thienyl)-1-propanone methanesulphonate, is reacted in the presence of a tertiary amine, preferably triethylamine, in the presence of formic acid, in the presence of p-cymenedichlororuthenium dimer and in the presence of (1S),(2S)-N-p-toluenesulphonyl-1,2-diphenylethylenediamine, to give (S)-3-N-methylarnino-1-(2-thienyl)-1-propanol.

The method according to the invention is carried out such that firstly the solvent and the compounds of formula (I) and the compounds of formula (II) are charged. Then, 3-N-methylamino-1-(2-thienyl)-1-propanone or/and salts thereof are added, preferably under inert gas, and the reaction mixture heated. The amine and the formic acid or/and the formate are then added, preferably metered in. The reaction may be accelerated by removal of the carbon dioxide formed, which is released during the reaction. The completion of the reaction may be determined by HPLC, for example. The reaction mixture may also be processed by methods known to those skilled in the art, e.g. by extraction or/and distillation.

Using the method according to the invention, (S)-3-N-methylamino-1-(2-thienyl)-1-propanol can be prepared in good yields and in high enantiomeric purities of up to 99.9%. The method according to the invention establishes for the first time the possibility of an efficient preparation of (S)-3-N-methylarnino-1-(2-thienyl)-1-propanol by means of an asymmetric transfer hydrogenation in industrial processes.

The following examples serve to exemplify the invention and are not regarded as limiting.

### EXAMPLES

### 1. Preparation of (S)-3-N-methylamino-1-(2-thienyl)-1-propanol starting from 3-N-methylamino-1-(2-thienyl)-1-propanone hydrochloride

77.5 g (0.76 mol) of triethylamine were initially charged at 5° C. 18.5 g (0.40 mol) of formic acid were then added in a temperature-controlled manner, such that the temperature did not exceed 20° C. 90.0 g (84% strength product, 0.37 mol) of 3-N-methylamino-1-(2-thienyl)-1-propanone hydrochloride were added and the reaction mixture diluted with 187 g of acetonitrile. To the reaction mixture were then added 2.0 g (22.9 mmol) of dimethylacetamide, 1.36 g (3.72 mmol) of (S,S)-Ts-DPEN, 1.14 g (1.86 mmol) of [(p-cymene)RuCl₂]₂ and 42.5 mg (0.42 mmol) of triethylamine. The reaction suspension was then warmed to 35-37° C. and stirred overnight at this temperature. 299.2 g of a 10.03% by weight solution of (S)-3-N-methylamino-1-(2-thienyl)-1-propanol (48%) in acetonitrile with an enantiomeric purity of >99.9% ee were obtained.

### 2. Preparation of (S)-3-N-methylamino-1-(2-thienyl)-1-propanol starting from 3-N-methylamino-1-(2-thienyl)-1-propanone methanesulphonate

0.456 g (1.25 mmol) of (S;S)-Ts-DPEN and 0.381 g (0.62 mmol) of [(p-cymene)RuCl₂]₂ were added to 260 g of methanol and the reaction mixture was stirred at 40° C. for 1 h. 53.9 g (0.15 mol) of 3-N-methylamino-1-(2-thienyl)-1-propanone methanesulphonate were added and a mixture of 9.2 g of triethylamine (0.09 mol) and 22.9 g of formic acid (0.50 mol) was metered in. In this process, the solid went into solution. The reaction solution was heated to 45° C. and stirred at 45° C. for 4 h. 0.456 g (1.25 mmol) of (S;S)-Ts-DPEN and 0.381 g (0.62 mmol) of [(p-cymene)RuC1₂]₂ were added afresh and the reaction solution was stirred a further 2 h. 278.5 g of a 3.36% by weight solution of (S)-3-N-methylamino-1-(2-thienyl)-1-propanol (48%) in acetonitrile with an enantiomeric purity of >99.9% ee were obtained.

### 3. Preparation of (S)-3-N-methylamino-1-(2-thienyl)-1-propanol starting from 3-N-methylamino-1-(2-thienyl)-1-propanone methanesulphonate with S,S-DPEN as Ligand (Comparative Example)

In a 100 mL round-bottomed flask equipped with magnetic stirrer, Liebig condensor and nitrogen line were placed 36.6 g of methanol, 77.7 mg (0.13 mmol) of [(p-cymene)RuCl₂]₂ and 67.3 mg (0.31 mmol) of S,S-DPEN. 5.00 g of 3-N-methylamino-1-(2-thienyl)-1-propanone methanesulphonate were then added and the reaction mixture was heated to 40° C. At 40-45° C., 1.2 g of triethylamine and 3.0 g of formic acid were then added and the reaction solution was stirred at 45° C. Reaction samples were taken after 23 hours and 47 hours. In this instance, no conversion to (S)-3-N-methylamino-1-(2-thienyl)-1-propanol occurred.

### 4. Preparation of (S)-3-N-methylamino-1-(2-thienyl)-1-propanol starting from 3-N-methylamino-1-(2-thienyl)-1-propanone methanesulphonate with S-Tol-BINAP as Ligand (Comparative Example)

In a 100 mL round-bottomed flask equipped with magnetic stirrer, Liebig condensor and nitrogen line were placed 36.6 g of methanol, 77.7 mg (0.13 mmol) of [(p-cymene)RuCl₂]₂ and 215 mg of S-Tol-BINAP. 5.00 g of 3-N-methylamino-1-(2-thienyl)-1-propanone methanesulphonate were then added and the reaction mixture was heated to 40° C. At 40-45° C., 1.2 g of triethylamine and 3.0 g of formic acid were then added and the reaction solution was stirred at 45° C. Reaction samples were taken after 23 hours and 47 hours. After 47 hours, a conversion of only 0.4% had been reached.

### 5. Preparation of (S)-3-N-methylamino-1-(2-thienyl)-1-propanol starting from 3-N-methylandno-1-(2-thienyl)-1-propanone methanesulphonate with S,S-Ts-DPEN as Ligand

In a 100 mL round-bottomed flask equipped with magnetic stirrer, Liebig condensor and nitrogen line were placed 36.6 g of methanol, 77.7 mg (0.13 mmol) of [(p-cymene)RuCl₂]₂ and 93 mg (0.25 mmol) of S,S-Ts-DPEN. 5.00 g of 3-N-methylamino-1-(2-thienyl)-1-propanone methanesulphonate were then added and the reaction mixture was heated to 40° C. At 40-45° C., 1.2 g of triethylamine and 3.0 g of formic acid were then added and the reaction solution was stirred at 45° C. Reaction samples were taken after 23 hours and 47 hours. Even after 23 h, a conversion of 80% had been achieved. After 47 hours, the conversion was 83%.

1. Method for preparing (S)-3-N-methylamino-1-(2-thienyl)-1-propanol and salts thereof, characterized in that 3-N-methylamino-1-(2-thienyl)-1-propanone or salts thereof, is reacted in the presence of at least one compound of formula (1)
[RuX₂(arene)]₂ (I)
where arene is an aromatic compound, bound to ruthenium in a co-ordinated manner, having 6 to 12 ring carbon atoms, which may be further substituted with up to 6 radicals, which independently of one another are selected from the group consisting of C₁-C₈-alkyl, benzyl and phenyl, and X is chlorine, bromine or iodine,
in the presence of at least one compound of formula (II) 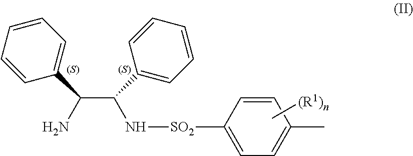
where R¹ can be the same or different and each is a residue from the series C₁-C₂₀-alkyl, C₁-C₂₀-alkoxy, C₁-C₂₀-fluoroalkyl, halogen, or C₆-C₁₅-aryl and n is 0, 1, 2, 3 or 4,
in the presence of at least one amine and
in the presence of formic acid or/and formates or mixtures thereof.

2. Method according to claim 1, characterized in that arene is mesitylene, cumene, p-cumene, o-cymene, m-cymene or benzene.

3. Method according to claim 1 or 2, characterized in that R′ is methyl, ethyl, n-, isopropyl, n-, sec-, tert-butyl or n-, sec-pentyl or fluorine or chlorine.

4. Method according to one or more of claims 1 to 3, characterized in that n =0 or 1, preferably n=0.

5. Method according to one or more of claims 1 to 4, characterized in that the amine is a tertiary amine, preferably triethylamine.

6. Method according to one or more of claims 1 to 5, characterized in that the method is carried out in the presence of a solvent selected from the group consisting of amides, nitriles or alcohols or mixtures of these solvents.

7. Method according to one or more of claims 1 to 6, characterized in that the molar ratio of formic acid to tertiary amine is from 3:1 to 0.8:1.

8. Method according to one or more of claims 1 to 7, characterized in that the method is carried out at a temperature of 30° C. to 60° C.

9. Method according to one or more of claims 1 to 8, characterized in that the molar amount of ruthenium used, based on the amount of 3-N-alkylamino-1-(2-thienyl)-1-propanone or salts thereof, is from 0.05 to 1 mol %.

10. Method according to one or more of claims 1 to 9, characterized in that the salt of the compound 3-N-alkylamino-1-(2-thienyl)-1-propanone used is the methanesulphonate or the hydrochloride.

11. Method according to one or more of claims 1 to 10, characterized in that (S)-3-N-methylamino-1-(2-thienyl)-1-propanol is prepared with an enantiomeric purity of more than 90%.
